(19) United States
(12) Patent Application Publication
(10) Pub. No.: US 2026/0007541 A1
(43) Pub. Date: Jan. 8, 2026

(54) NON-ADHESIVE PERISTOMAL SEAL ASSEMBLY

(71) Applicants: Kalin Owens, Fayetteville, AR (US); Kody Owens, Springdale, AR (US)

(72) Inventors: Kalin Owens, Fayetteville, AR (US); Kody Owens, Springdale, AR (US)

(21) Appl. No.: 19/249,505

(22) Filed: Jun. 25, 2025

Related U.S. Application Data
(60) Provisional application No. 63/666,827, filed on Jul. 2, 2024.

(51) Int. Cl. A61F 5/448; A61F 5/449

(52) U.S. Cl. CPC A61F 5/448; A61F 5/449

(57) ABSTRACT

Apparatus and method for supporting an ostomy bag used by an ostomate having a stoma. A flexible seal insert has opposing first and second annular flanges and an intermediate neck portion to define a central passageway configured to extend about the stoma. The first annular flange sealingly engages a peristomal area at the base of the stoma. A second annular flange extends through an opening in the ostomy bag to provide back flow retention of contents within the bag. The seal insert extends through a retention plate to which the ostomy bag is attached using a wax ring and adhesive layer. A belt secures the plate to a torso of the ostomate. The intermediate neck area protects the stoma from an edge of the bag opening, and the first annular flange is held in place without the need to apply adhesives or other irritants to the peristomal area.

### RELATED APPLICATIONS

The present application makes a claim of domestic priority under 35 U.S.C. 119(e) to co-pending U.S. Provisional Patent Application No. 63/666,827 filed Jul. 2, 2024, the contents of which are hereby incorporated by reference.

### BACKGROUND

In the field of medicine, a stoma is an opening that a surgeon creates in an abdomen of a patient (ostomate) to facilitate the external passage of fluids from the patient's digestive tract. An ostomy procedure connects a portion of the intestines or bladder to the abdominal wall of the patient, allowing an end portion of the intestines (the “stoma”) to project outwardly. An ostomy pouch (bag) is often used to collect the fluids (bile, urine, fecal waste, etc.) that emit from the stoma.

A variety of ostomy pouch configurations are known and are commercially available for use by ostimates. Many pouch designs tend to have a central opening that may be cut to size to fit over and surround the stoma. An annular ring of wax or similar fluidic seal surrounds the central opening, and an annular adhesive strip surrounds the wax seal.

Surface preparations are applied to the skin surrounding the stoma (the “peristomal area”), and the wax seal and the adhesive strip are thereafter directly applied to the peristomal area. The adhesive strip secures the pouch in place, and the wax seal nominally establishes a fluidic seal to retain spillage from the pouch that flows to the peristomal area at the base of the stoma.

While operable, these and other existing stoma pouch applications present a number of disadvantages. Because it is often necessary to remove and replace filled pouches on a regular basis, the peristomal area can become irritated, inflamed and even ulcerated due to the repetitive cleaning and reapplication of adhesive and other materials. Moreover, applications that directly apply the ostomy pouch to the peristomal area can cause discomfort or leakage due to movement and exertion by the patient.

There is therefore a continual need for improvements the art to address these and other limitations with existing ostomy pouch systems. It is to these and other improvements that various embodiments of the present disclosure are generally directed.

### SUMMARY

Various embodiments of the present disclosure are generally directed to systems and methods for supporting an ostomy pouch (bag) used by an ostomate having a stoma.

In some embodiments, a flexible seal insert has opposing first and second annular flanges and an intermediate neck portion to define a central passageway configured to extend about the stoma. The first annular flange sealingly engages a peristomal area at the base of the stoma. the second annular flange extends through an opening in the ostomy bag to provide back flow retention of contents within the bag. The intermediate neck area protects the stoma from an edge of the bag opening.

In further embodiments, the seal insert extends through a retention plate to which the ostomy bag is attached using a wax ring and adhesive layer of the ostomy bag, and a belt secures the retention plate to a torso of the ostomate. In this way, the first annular flange is held in place without the need to apply adhesives or other irritants to the peristomal area.

The various embodiments can be used with any number of commercially available ostomy bags, including one-piece and two-piece ostomy bag assemblies.

These and other features and advantages of various embodiments can be understood from the following detailed description in conjunction with a review of the accompanying drawings.

### BRIEF DESCRIPTION OF DRAWINGS

FIG. 1 is an exploded isometric view of a non-adhesive peristomal sealing system (appliance) constructed and operated in accordance with some embodiments of the present disclosure.

FIGS. 2A through 2D show various views of a seal insert of the system of FIG. 1 in some embodiments.

FIG. 2E shows another seal insert with a smaller interior diameter that can alternatively be used in the system of FIG. 1 in further embodiments.

FIGS. 3A and 3B show further details of the seal insert in accordance with further embodiments.

FIGS. 4A through 4D show aspects of a plate assembly of the system of FIG. 1 in accordance with some embodiments.

FIG. 5 is a schematic depiction of a one-piece ostomy pouch (bag) assembly in accordance with the related art suitable for use with the system of FIG. 1.

FIG. 6 is a schematic depiction of a two-piece ostomy pouch assembly in accordance with the related art that is also suitable for use with the system of FIG. 1.

FIG. 7A shows a cross-sectional, elevational view of the assembled system of FIG. 1 using the one-piece ostomy pouch of FIG. 5 in some embodiments.

FIG. 7B shows the arrangement of FIG. 7A secured to a patient.

FIG. 7C shows another arrangement of the assembled system of FIG. 1 using the two-piece ostomy pouch of FIG. 6 and omitting the plate 118 of FIG. 1 in further embodiments.

FIG. 8 is a sequence diagram to depict steps that can be carried out to assemble, install and remove an assembled system in accordance with further embodiments.

FIG. 9 is a schematic depiction of another non-adhesive peristomal sealing system in accordance with further embodiments in which an optional fluid barrier member is utilized between the stoma and the seal insert.

FIG. 10 is a schematic depiction of yet another non-adhesive peristomal sealing system in accordance with further embodiments in which an inner surface of the seal insert is sized to contactingly engage an outer sidewall of the stoma.

### DETAILED DISCUSSION

Various embodiments of the present disclosure are generally directed to systems and methods for securing an ostomy pouch without the need for the use of adhesives or other layers of sealing and/or securement material against a peristomal area of a patient (ostomate).

As explained below, some embodiments are directed to a seal system (appliance) having a plate assembly and a specially configured seal insert which cooperate to secure the ostomy pouch to the patient. The plate assembly includes a belt and buckle arrangement or similar to engage a retention plate having a central aperture to accommodate passage of the seal insert.

Any number of commercially available ostomy pouches may be used. Instead of attaching the pouch directly to the body, the pouch is secured to the sealing plate using provided adhesives and sealing layers of the pouch, and the plate holds the seal insert against the ostomate's body.

The seal insert has opposing first and second ends and an intervening body (neck) portion extending therebetween. The first end has a seating surface (first flange) that contactingly engages and establishes a fluidic seal against the skin of the patient. The neck portion of the seal insert extends from the first end through the central aperture of the plate and into an opening in the pouch.

The second end of the seal insert includes an outwardly projecting, annular lip (second flange) that extends into the pouch. The diameter of the second flange is larger than the opening in the bag, thereby serving as a back flow retention flange that reduces back flow of the contents of the pouch out of the pouch and onto the patient in the peristomal area. The seal insert is made of a flexible material such as medical grade silicone, allowing the second end of the flange to be easily deformed for passage through the pouch opening.

In some embodiments, the seal insert surrounds the stoma of the patient in a non-contacting, proximal relation thereto, so that there is an annular clearance gap between an inner sidewall of the neck portion of the seal insert and an outer sidewall of the stoma. In further embodiments, a fluid barrier member is contactingly disposed between the stoma and the seal insert to partially or fully fill the intervening gap between the seal insert and the stoma. In still other embodiments, the seal insert is sized to contactingly engage the sidewall of the stoma.

The system eliminates the need to apply adhesives, seals, sealant pastes or other materials directly to the sensitive skin around the stoma in the peristomal area. In existing systems, these adhesives and pastes must generally be scrubbed clean from around the stoma to prepare for the next stoma bag application. As noted previously, the repetitive application and removal of these materials can cause irritation and injury to the patient over time.

While lubricating or other therapeutic materials may be optionally used to interface between the seal insert and the skin of the patient, it is contemplated that no such materials are necessary or desired. Instead, the first end directly, compressingly engages the skin without the need for a separate sealing and/or adhesive layer to secure the appliance to the patient or maintain a fluidic seal.

Because the seal insert is formed of a soft, non-irritating material, the seal insert can be comfortably worn directly against the patient's skin. Nevertheless, it has been found in some cases that the application of a barrier spray to the seal and skin interface before applying the seal and belt can supply further protection against irritation and leakage. It will be appreciated that skin barrier sprays and wipes have no adhesive properties and tend to increase lubrication between the seal insert and the patient, rather than attachment of the seal insert to the patient.

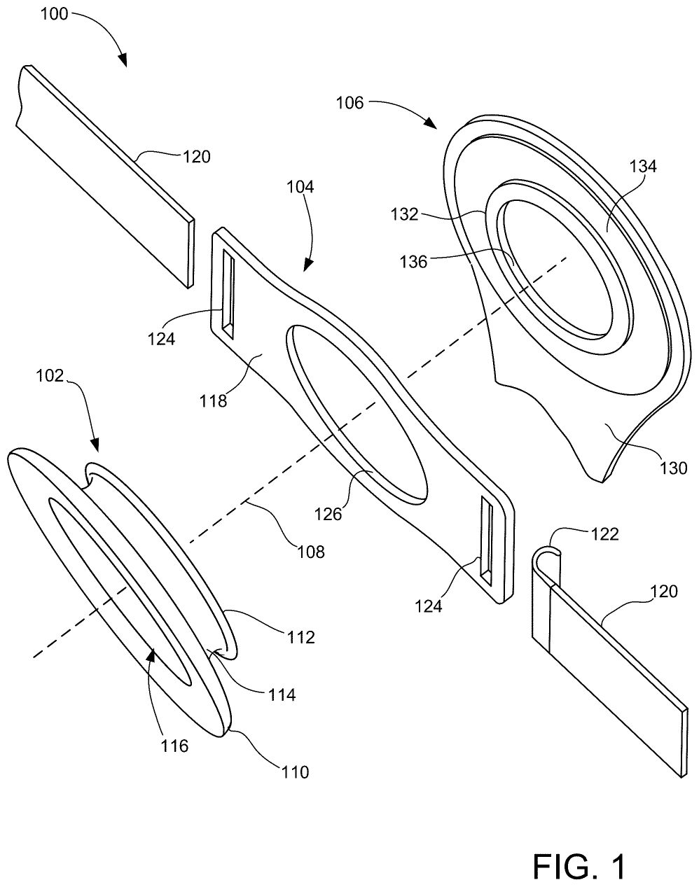

These and other features and advantages of various embodiments can be understood beginning with a review of FIG. 1 which provides an exploded isometric representation of a non-adhesive peristomal sealing system (appliance) 100. The system 100 includes a flexible seal insert 102, a plate assembly 104, and an ostomy pouch assembly 106 (also referred to sometimes as an ostomy bag). These various elements are aligned along a central axis 108.

The seal insert 102 is formed of any number of suitable soft and non-reactive materials such as but not limited to medical grade silicone. The seal insert 102 includes opposing first and second ends 110, 112 separated by a tapered intermediate neck portion 114. A central aperture 116 extends through the seal insert as shown.

The plate assembly 104 includes a flexible backing plate 118 and a belt 120. The plate 118, also sometimes referred to as a retention plate, is secured using one or more buckles 122 or other fastening mechanisms of the belt 120 to attach the plate 118 about the torso of the patient (ostimate). Slotted apertures 124 can be provided to accommodate the belt material and/or the buckles. For example, one end of the belt 120 may be looped and stitched to one side of the plate 118, and the buckle 122 can engage the other side of the plate. The belt 120 may be formed of elastic material, although inelastic belts and adjustable length buckle arrangements can be used, as well as other belt arrangements as required.

A central plate aperture 126 extends through a medial extent of the plate 118 to accommodate the second end 112 and the neck portion 114 of the seal insert 102. Any number of suitable materials can be used for the plate including metal, plastic, etc. As explained below, in at least some embodiments the plate is sufficiently rigid to sealingly engage the pouch assembly 106 while being sufficiently flexible to conform to the body of the patient and facilitate movement thereof while retaining pressing, sealing engagement of the seal insert 102 thereagainst.

The pouch assembly 106 includes a pouch or bag portion 130 made of fluid-tight flexible material adapted to accumulate and retain a selected volume of discharge fluid from the patient. Sealing and adhesion materials extend from the pouch 130 including a sealing ring 132 and an adhesive layer 134. In one example, the sealing ring 132 may have a wax or similar base and is adapted to generate a fluidic seal, and the adhesive layer may be pressure sensitive tape or similar and is adapted to provide mechanical attachment and securement of the pouch assembly 106. The ring and adhesive layer 132, 134 surround a central pouch aperture 136.

In a conventional application of the pouch assembly 106, the ostomate cuts through a layer of material to match the pouch aperture 136 to the size of the stoma, slides the pouch 130 onto the stoma, and presses the ring 132 and tape 134 directly against the skin in the peristomal area at the base of the stoma.

In the system 100 disclosed herein, however, the pouch aperture 136 is cut to maximum size (e.g., up to the inner diameter of the wax ring 132), the seal insert 102 is passed through the central aperture 126 of the plate and through the pouch aperture 136 into the pouch 130, the wax ring 132 and tape 134 are pressed against the plate 118, and the lower end 110 of the seal insert 102 is pressed against the ostomate. Other arrangements can be used as discussed below.

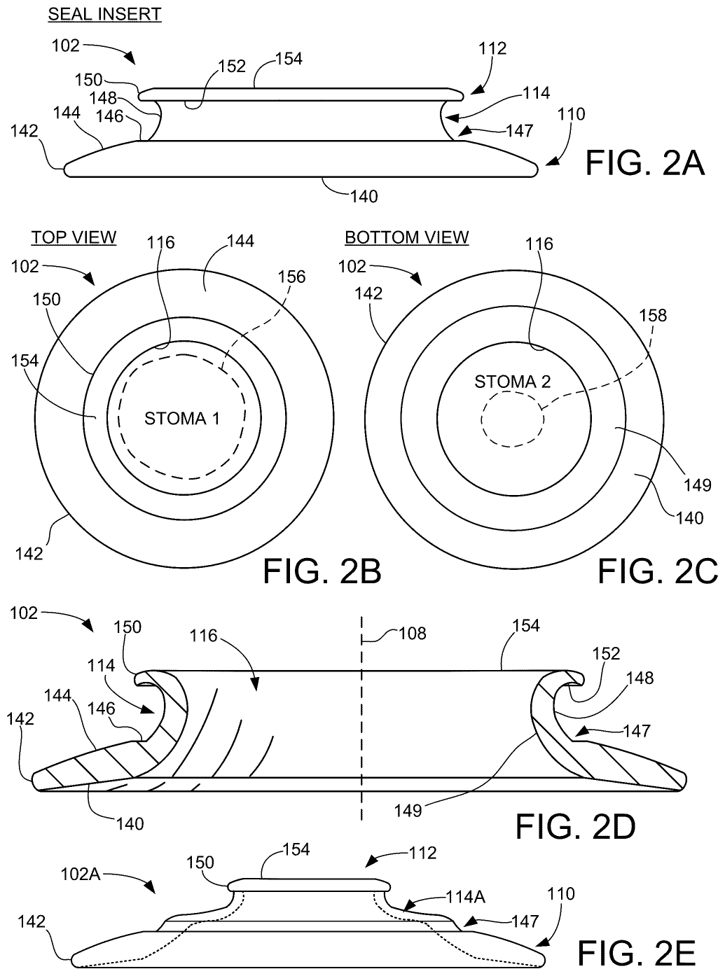

FIGS. 2A through 2D show various views of the seal insert 102 from FIG. 1 in accordance with some embodiments. While a generally contoured, axially symmetric shape is shown, non-axially symmetric configurations can be used such as oval, etc. As such, FIGS. 2A-2D are merely illustrative and not limiting.

The first end 110 of the seal insert 102, also sometimes referred to as a lower end, takes an annular flange configuration with a lower contact surface 140 adapted to contactingly and compressingly engage the peristomal area (e.g., skin area surrounding the stoma). In a normally relaxed state, the surface 140 is generally frusto-conically shaped to as to extend upwardly at a desired angle from a base plane. This contoured shape provides a pre-bias that helps to ensure flat engagement when the seal insert 102 is installed. The first end 110 is thus also sometimes referred to as a first flange or a sealing flange.

An outer perimeter of the lower end 110 is denoted at 142 and has a radiused shape as shown. An upper contact surface 144 of the lower end 110 extends in opposition to the lower contact surface 140. While the respective surfaces 140, 144 diverge so that the seal insert material gains in thickness in a direction moving away from the outer perimeter 142, other arrangements can be used including an arrangement in which the respective surfaces 140, 144 are parallel, tend to diverge, etc.

The upper contact surface 144 terminates in an annular shelf surface 146. The shelf surface 146 is relatively narrow and surrounds a base portion of the intermediate neck portion 114. An annular corner 147 is formed at the junction of the horizontal shelf surface 146 and an upwardly extending, exterior sidewall 148 of the intermediate portion 114.

The corner 147 is configured to contactingly engage the corner edge of the plate 118 to form a fluidic seal between the plate and the seal insert 102; more particularly, the flat planar extent of the plate 118 surrounding the central aperture 126 will contactingly engage the shoulder surface 146, and the sidewall of the central aperture 126 will contactingly engage the lower portion of the exterior sidewall 148. This corner engagement will also help maintain a flattened orientation of the lower end 110 against the patient.

As best viewed in FIG. 2D, the intermediate portion 114 is bounded by the curvilinearly extending exterior sidewall 148 as well as a corresponding, curvilinearly extending interior sidewall 149. Both sidewalls 148, 149 are convex with respect to the central axis 108 and taper inwardly in a direction toward the axis 108. Other sidewall configurations can be used.

Referring again to FIG. 2A, the second (upper) end 112 is also characterized as having an annular flange configuration with an outwardly extending retention lip 150 at an outermost perimeter thereof defined by opposing lower and upper curvilinearly extending sidewall surfaces 152, 154. The lip 150 generally aligns with the outermost edge of the shoulder surface 146, but other axial alignment configurations can be used. The lip 150 is also sometimes referred to as a second annular flange or a back flow retention flange.

With reference to the respective top and bottom views of FIGS. 2B and 2C, the central passageway 116 is sized to accommodate a variety of differently sized stomas, including a relatively larger diameter stoma as represented at 156 (“STOMA 1”) and a relatively smaller diameter stoma as represented at 158 (“STOMA 2”). Without limitation, STOMA 1 may be on the order of about 2 inches, in. (5.08 centimeters, cm) in diameter, and STOMA 2 may be on the order of about 1 in. (2.54 cm) in diameter. As will be recognized, the size of a particular stoma will depend upon a number of factors including the physiology of the patient, the location at which the stoma is formed, whether the stoma includes a single opening or multiple openings, etc. Nonetheless, most stomas will tend to fall within the 1-2 in. diameter range, and will be largely cylindrically shaped.

Accordingly, the seal insert 102 is designed to provide efficient sealing and support for patients with substantially any sized stoma. For example, the opening 116 in FIG. 2B is approximately 2.44 in. (6.35 cm) in diameter so as to be closely positioned in non-contacting relation to the larger STOMA 1 156.

While this larger opening 116 is also effective for smaller stomas such as STOMA 2 158, FIG. 2E shows an alternative seal insert 102A with a greater amount of taper in the intermediate portion (denoted at 114A) to provide a smaller interior opening, such as on the diameter of about 1.25 in. (3.18 mm), to more closely surround the smaller STOMA 2. It will be noted that the corner junction 147 is provisioned as before to accommodate the corner of the plate (118, FIG. 1).

While not shown in FIGS. 2A-2E, in further embodiments a cushioning collar or other fluidic barrier member can be inserted in the annular gap between the stoma and the sidewall 149 to further fluidically separate the patient from the contents of the pouch assembly 106 and provide non-irritating contact and support upon the outer annular sidewall of the stoma.

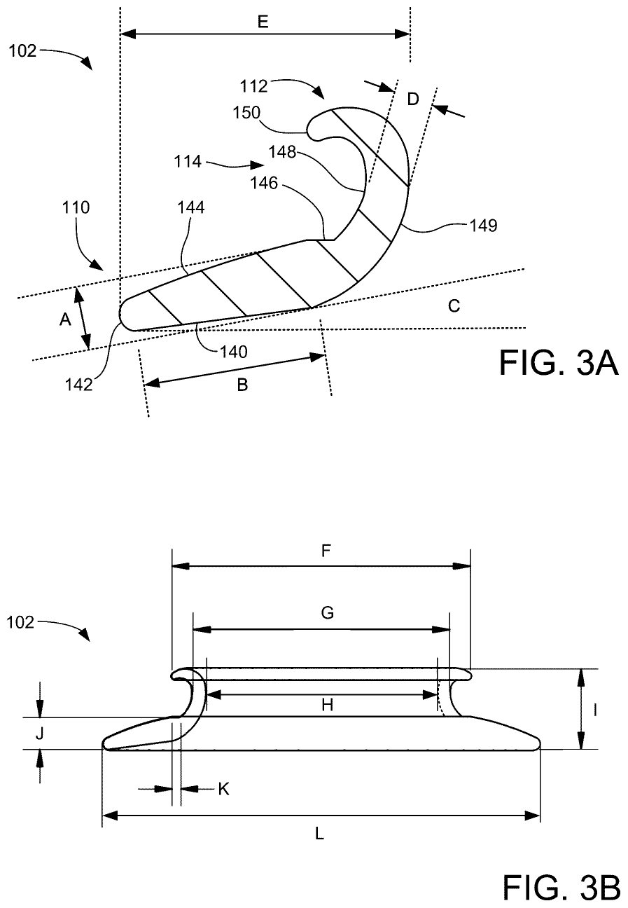

FIG. 3 provides a detailed representation of the cross-sectional portions of the seal insert 102 from FIG. 2D in accordance with some embodiments. While the particular dimensions of a given configuration of seal insert will depend upon the requirements of the patient application, Table 1 below provides exemplary values for the features denoted in FIG. 3A:

TABLE 1

| Interval | Feature | Value | |
|---|---|---|---|
| A | Maximum Localized Wall Thickness | 0.25 in. | (63.5 mm) |
| B | Lower Surface Radial Length | 0.688 in. | (174.6 mm) |
| C | Projection Angle | 7.5 degrees | |
| D | Maximum Localized Wall Thickness | 0.125 in. | (31.8 mm) |
| E | Radial Length | 1.06 in. | (27.0 mm) |

FIG. 3B is a composite representation of the views of the seal insert 102 in FIGS. 2A and 2D. Table 2 provides additional exemplary dimensions for various features denoted therein. As before, these dimensions are merely exemplary:

TABLE 2

| Interval | Feature | Value | |
|---|---|---|---|
| F | Retention Lip Width | 3.19 in. | (8.10 cm) |
| G | Neck Outermost Diameter | 2.75 in. | (6.99 cm) |
| H | Central Aperture Diameter | 2.44 in. | (6.19 cm) |
| I | Overall Seal Insert Height | 0.88 in. | (22.3 mm) |
| J | Height of Shoulder Surface | 0.31 in. | (7.87 mm) |
| K | Shoulder Surface Radial Length | 0.0625 in. | (1.59 mm) |
| L | Overall Seal Insert Diameter | 4.625 in. | (11.75 cm) |

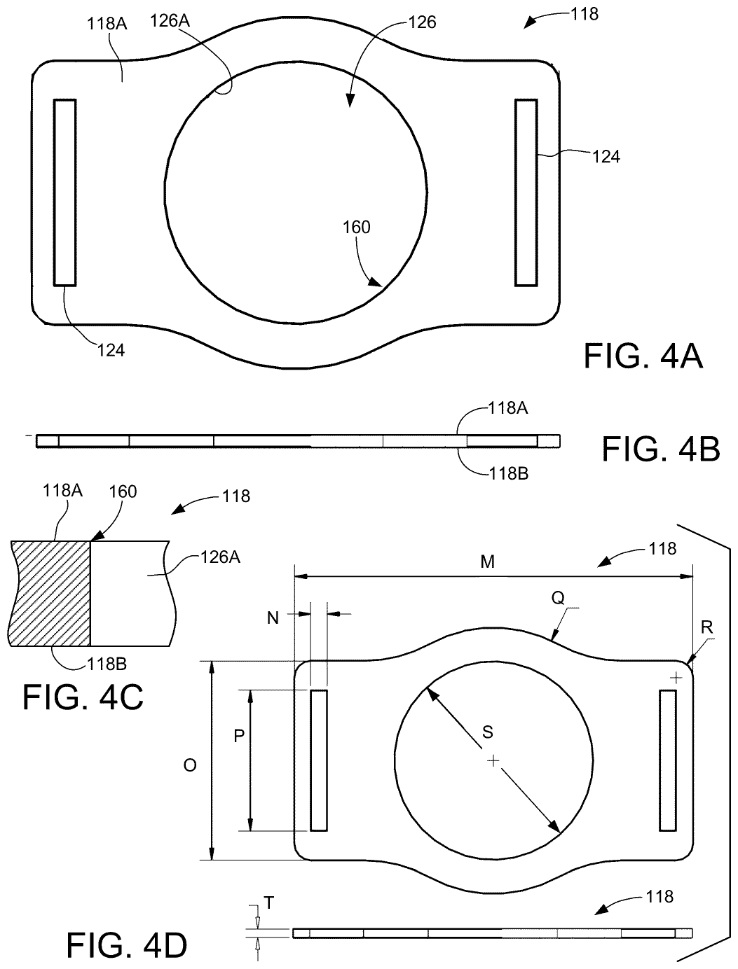

FIGS. 4A through 4D depict the plate 118 from FIG. 1 in some embodiments. Other configurations can be used. As noted previously, the plate 118 provides a downwardly directed, compressive bias force upon the seal insert 102. To this end, the plate 118 is provided with opposing planar surfaces 118A, 118B (see FIG. 4B). As best shown in FIG. 4C, an inner corner 160 extends at the junction of the edge of planar surface 118A and central aperture sidewall 126A to contactingly mate with the corner junction 147 of the seal insert (see FIGS. 2A, 2D).

Table 3 shows various exemplary dimensions for the plate 118. As before, other dimensions can be used:

TABLE 3

| Interval | Feature | Value | |
|---|---|---|---|
| M | Overall Length of Plate | 6.0 in. | (15.24 cm) |
| N | Bracket Aperture Width | 0.25 in. | (6.35 mm) |
| O | Central Aperture Diameter | 2.44 in. | (6.19 cm) |
| P | Plate End Width | 3.0 in. | (7.62 cm) |
| Q | Outer Radius of Curvature | 4.0 in. | (10.16 cm) |
| R | Corner Radius of Curvature | 0.25 in. | (6.35 mm) |
| S | Interior Diameter of Central Aperture | 3.0 in. | (7.62 cm) |
| T | Thickness of Plate | 0.0625 in. | (1.59 mm) |

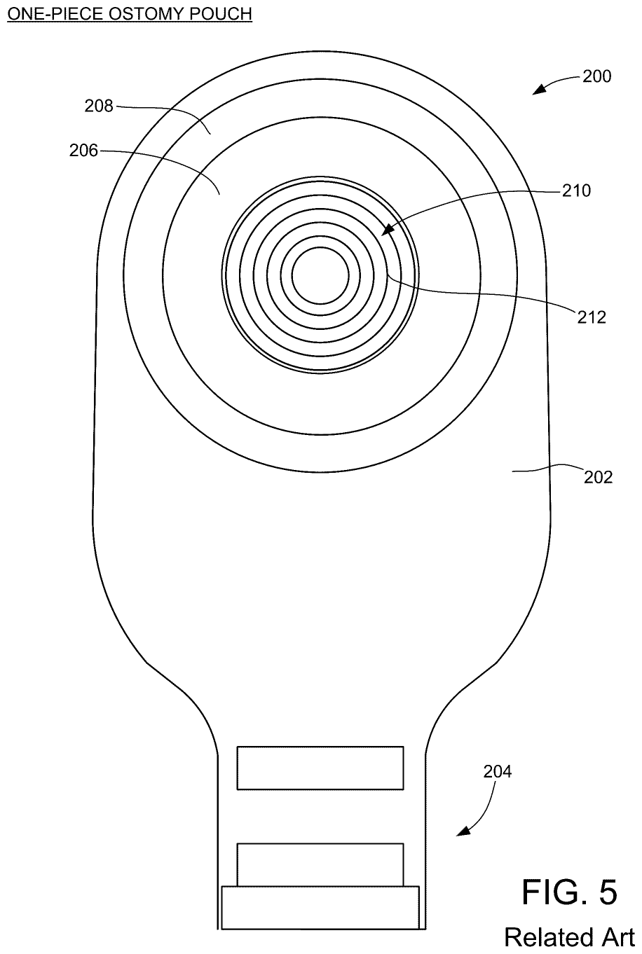

FIG. 5 shows a one-piece ostomy pouch assembly 200 of the related art. The pouch assembly 200 is similar to the pouch assembly 106 depicted in FIG. 1 and includes a fluid impermeable pouch (bag) portion 202 configured to collect and retain fluids emitted by the stoma. The assembly 200 is shown to be reclosable via reclosable assembly 204. Reclosable bags can take a variety of forms and can include adhesive, hook and loop fasteners, and other mechanisms to allow the patient to controllably open and release (drain) the contents of the bag without necessarily requiring the bag be removed from the patient.

The assembly 200 further includes an annular scaling ring 206 and an annular adhesive layer 208 which surrounds the ring 206. The ring 206 and adhesive layer 208 generally correspond to the elements 132, 134 in FIG. 1 and serve to attach the bag 202 to the plate 118 as before.

A removable substrate 210 extends across the opening (not shown) in the bag. This substrate is formed of paper, fiber board or similar material. Printed indicia 212 is provided in the form of concentric circles at varying radii about a center point 214 of the opening. It will be appreciated that the center point 214 nominally aligns with the central axis 108 in FIG. 1.

As will be recognized, pouch assemblies such as 200 are typically provided with directions that direct the patient to determine the outermost diameter of their stoma and then, with a pair of scissors or other cutting tool, cut an opening through the substrate 210 to surround and admit passage of the stoma thereto. While operable, it is difficult to cut a clean aperture, so that the opening may be irregular (jagged, etc.). This provides a number of disadvantages, including the possibility of irritation to the sidewalls of the stoma, etc.

By contrast. the system 100 can readily be used with pouch assemblies such as 200 by having the user cut the substrate 210 to the maximum permittable size (e.g., around 2.5 inches in diameter) to allow passage of the intervening seal insert through the formed opening and into the pouch 202. The neck portion 114 of the seal insert 102 is thus interposed between the stoma and the bag, thereby protecting the outer annular sidewall of the stoma from the rough, jagged edges of the opening.

It will be appreciated that other forms of bags can be used, including bags that are precut to a particular size of opening without the need for the ostomate to cut the opening. Similarly, which presently discussed embodiments size the seal insert and opening to accommodate a maximum opening size, this is merely exemplary and is not limiting. So long as the neck portion of the seal insert is smaller than the bag opening and the back flow flange is larger than the bag opening, and both are sized to accommodate the stoma, the system will work effectively. Any number of different combinations for these various parameters can be accommodated for a given patient application.

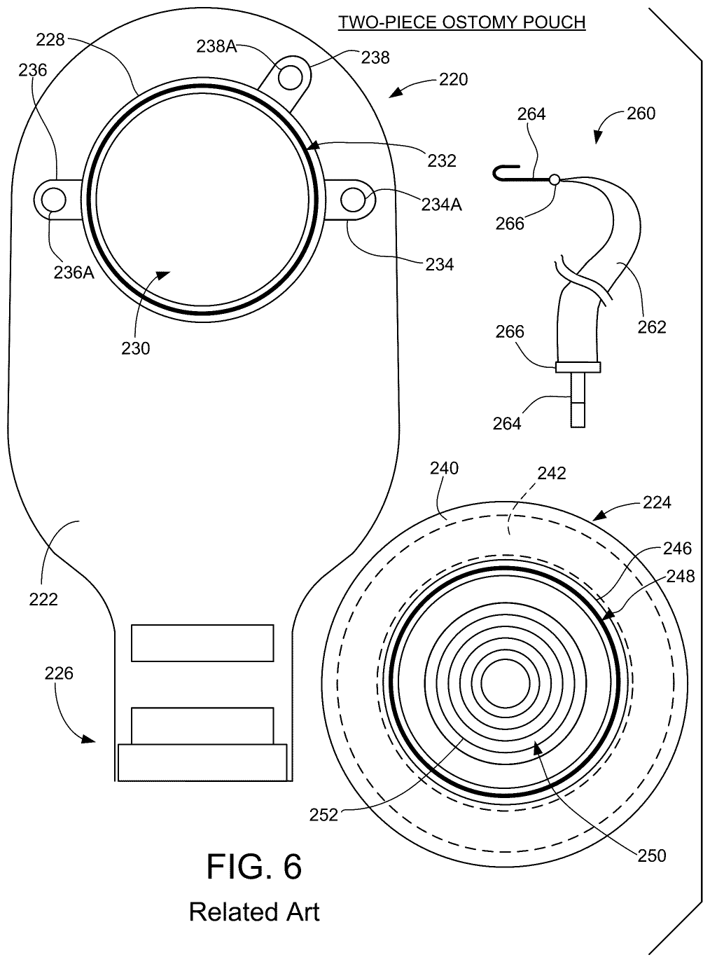

FIG. 6 shows a two-piece ostomy pouch assembly 220 in accordance with the related art. The pouch assembly 220 is also suitable for use with the system 100 of FIG. 1 and generally corresponds to the pouch assemblies 106, 200 described above. In this case, the assembly 220 has a pouch (bag) 222 that is designed to be attached to and removed from a separate attachment portion 224 that is affixed to the patient.

To this end, the bag 222 has an optional reclosable assembly 226 at the lower extent of the bag which allows draining of the contents as before. The bag 222 further has a ring shaped seal and support member 228 at an upper extent of the bag, which is permanently affixed to the bag and surrounds a bag aperture 230.

The ring shaped member 228, also sometimes referred to as a first ring member, is formed of rigid plastic or similar material and includes an annular groove 232. Scurement tabs 234, 236 and 238 extend radially from an outer peripheral extent of the first ring member 228, and have respective securement tab apertures 234A, 236A and 238A.

The attachment portion (member) 224 is designed to be directly attached to the skin of the patient in the peristomal area and serves as a coupling for the bag portion 222. To this end, the attachment member 224 has a substrate 240 that spans the entirety of the member 224 and provides a support surface for the various elements attached thereto. One such element is one or more annular layers of sealing and adhesive material depicted generally at 242. It will be noted that the sealing and adhesive material 242 can include a wax ring, adhesive tape, or other suitable materials as before, and is located on the underside of the substrate 240.

Disposed within the annular extent of the material 242 is a second ring shaped member 246. The second ring shaped member 246, also sometimes referred to as a second ring member, has a corresponding annular tab or similar insertion member 248. The insertion member 248 in the second ring member 246 is designed to matingly engage the groove 232 in the first ring member 228.

As before, the substrate 246 may be formed of paper, cloth or other fiberous material, etc. An interior portion 250 of the substrate 246 is provided with printed indicia 252 to allow the patient (or other user) to cut an appropriately sized opening therethrough to accommodate the stoma.

Once this opening has been cut, the patient is directed during a conventional installation sequence to attach the attachment member 224 directly to the peristomal area of the patient using the material 242. The patient next places the first ring shaped member 232 of the removable bag 220 into mating engagement with the second ring shaped member 246. It will be appreciated that any number of sealing interfaces can be used to interconnect the bag 220 to the attachment member 224 and form a fluidic seal therebetween.

To support the weight of the bag 222, a belt assembly 260 can be optionally provided. The belt assembly 260 may be supplied with the bag 222 and the attachment mechanism 224, or may be provided separately. The belt assembly 262 includes an elongated strap portion 262 configured to encircle the torso of the patient. The strap portion 262 is usually elastic, but this is not necessarily required. A buckle (e.g., a hook, etc.) 264 is attached via a swivel or other attachment mechanism 266 to each end of the strap portion 262. The buckles 264 can engage selected ones of the tab apertures 234A, 236A, 238A of the first ring member 228 to provide further mechanical support for the bag 222.

Two-piece pouch assemblies such as 220 can be used with various embodiments of the present system 100. In one contemplated embodiment, the attachment member 224 is attached to the plate 118 as before, and the bag 222 is mated to the attachment member 224, so that the two-piece ostomy pouch assembly 220 is attached essentially the same way as for the one-piece ostomy pouch assembly 200. The belt 120 (see FIG. 1) secures the plate 118 to the patient and the belt assembly 260 in FIG. 6 remains unused.

In another contemplated embodiment, the plate 118 is eliminated, the attachment member 224 is attached directly to the seal insert 102, and the first ring member 228 and the second ring member 246 in combination serve as the plate to secure and compress the seal insert 102 against the patient. In this alternative arrangement, the belt assembly 260 is used to attach the plate (e.g., first ring member 228) to the patient to form the requisite plate assembly.

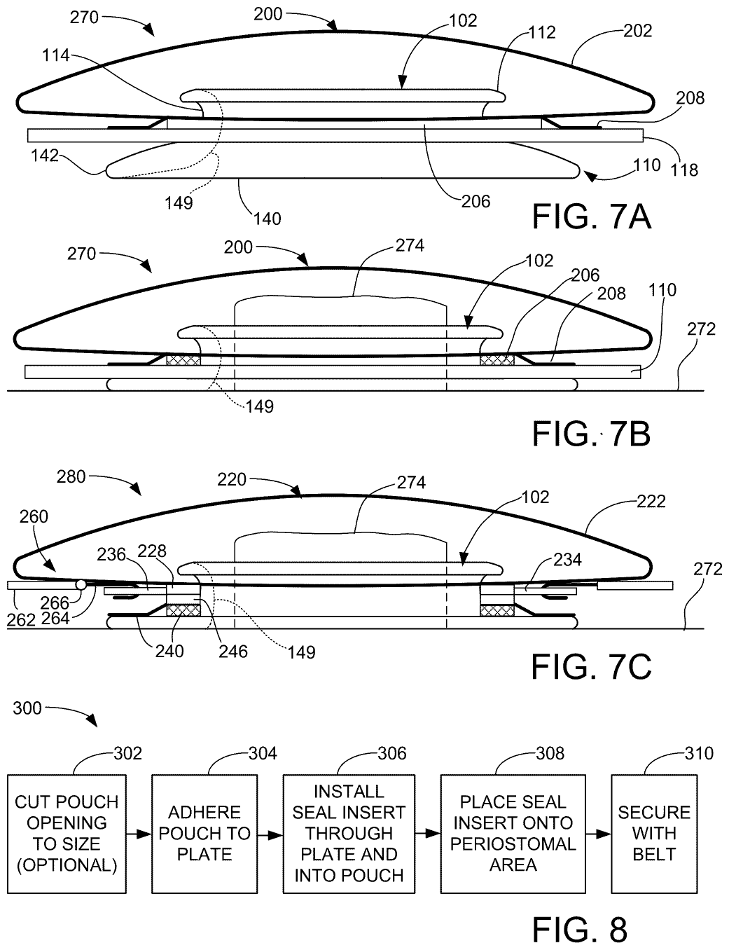

FIG. 7A shows an assembled system 270 using the one-piece pouch assembly 200 from FIG. 5. The system 270 is similar to the system 100 in FIG. 1 and is located in a position separate from the patient (e.g., prior to installation of the appliance). The respective interrelation of the seal insert 102, plate assembly 104 and pouch assembly 200 includes attachment of the sealing and adhesive layers 206, 208 from FIG. 5 to the plate 118; contacting engagement of the plate 118 against the seal insert 102; and extension of the second end of the seal insert into the bag 202. The interior sidewall 149 (FIG. 2D) is generally represented in dotted line fashion.

FIG. 7B shows the system 270 from FIG. 7A upon installation against the body of the patient (denoted at 272). A representative stoma is denoted at 274. At this point it will be noted that the taper supplied to the lower end of the seal insert 102 allows the bottom surface 140 of the seal insert (FIG. 2D) to lay flat against the patient surface 272, and provide a uniform pressure distribution thereagainst.

FIG. 7C shows an alternative system 280 in which the two-piece pouch assembly 220 from FIG. 6 is secured to the patient using the seal insert 102. In this embodiment, the attachment member 224 has been attached directly to the exterior base surfaces of the seal insert 102 using the adhesive and sealing layers 240; the plate 118 has been eliminated; and the first and second ring members 228, 246 (see FIGS. 5 and 6) and belt assembly 260 (FIG. 6) secure the seal insert 102 against the patient 272.

FIG. 8 provides a sequence flow diagram 300 to summarize various steps carried out by a patient in installing and removing the system 100 in accordance with the foregoing discussion. It will be appreciated that other steps may be used as required. It is contemplated that this sequence will include the use of the plate 118 with either a one-piece or two-piece pouch assembly. Similar operations can be carried out when the ring members/belt of a two-piece pouch assembly are utilized and the plate 118 is omitted.

At block 302, if the provisioned pouch assembly 106, 200, 220 is initially closed, an opening is cut to an appropriate size. As discussed previously, the size of the opening will be to accommodate the outer surface 146 of the seal insert, so that the seal insert presses against the interior sidewall of this opening in sealing engagement therewith.

At block 304, the pouch assembly is adhered to the plate, such as via the various sealing and adhesive materials 132, 134, 206, 208, 242 discussed above. The seal insert is next inserted through the central opening of the plate and into the opening in the pouch at block 306. As required, the flexible seal insert material may need to be deformed (squeezed) to allow the retention lip to pass through the respective openings. At this point, the assembly will generally resemble the unattached configuration of FIG. 7A.

At block 308, the assembled appliance is gently lowered onto the patient so that the seal insert comes into contacting engagement against the peristomal area surrounding the stoma. The patient then holds the applicance in place with one hand, while guiding the belt around the torso to secure the appliance in place, block 310. It will be noted that another advantageous function of the retention lip is that this tends to retain the appliance in an assembled state during installation due to the retention lip remaining retained within the bag opening.

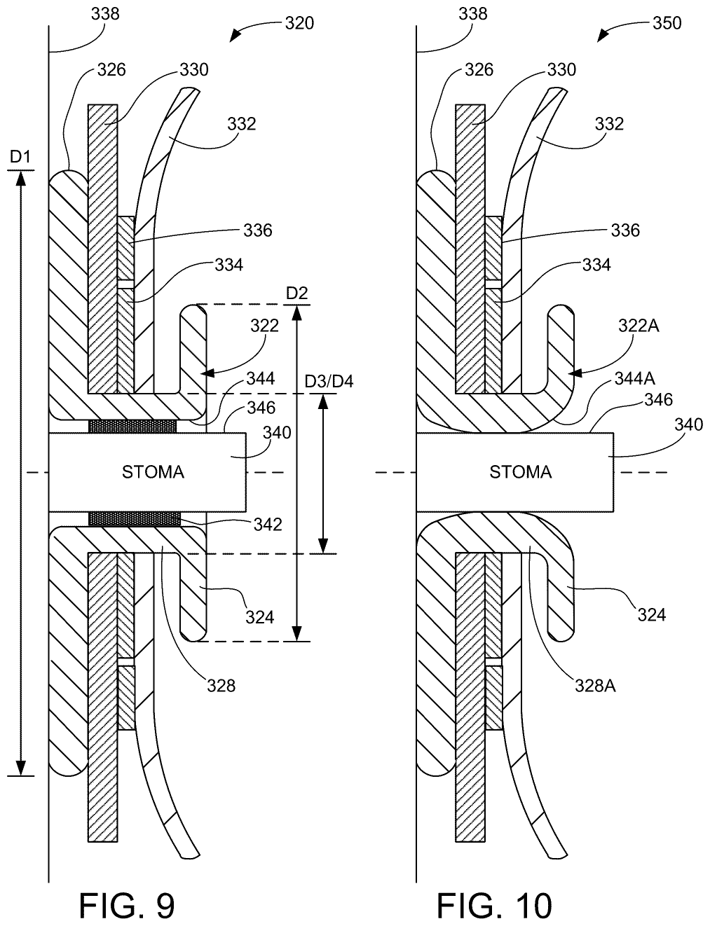

FIG. 9 is a simplified schematic representation of another system 320 similar to the systems shown above. Various features and sealing interfaces are noted, including a seal insert 322 with upper end flange 324, lower end flange 326, tapered intermediate neck portion 328, securement plate 330, bag 332, annular wax seal 334 and annular strip of adhesive tape 336. The system is mounted to a torso 338 of a patient having a stoma 340.

In FIG. 9, a fluid barrier member 342 is inserted between an inner annular sidewall 344 of the intermediate neck portion 328 and an outer annular sidewall 346 of the stoma 340. The fluid barrier member 342 can take a variety of forms including a soft absorbent material such as cloth, foam, etc., or a soft non-absorbent material such as silicone, wax, rubber, etc. The fluid barrier member 342 is configured to substantially fill the gap between the respective sidewalls 344, 346 and reduce leakage from the bag 332 onto the torso 338 of the patient in the peristomal area.

From FIG. 9, various relative sizes of different elements can be demonstrated. For example, the lower end (first annular) flange 326 has a first overall diameter D1, which is greater than a second overall diameter D2 of the upper end (second annular) flange 324. While these respective sizes can vary, it will be noted that D1 is generally sufficiently large to accommodate the attachment of the ostomy bag 332 such as via wax ring 334 and adhesive layer 336, while D2 is small enough to fit through the opening in the ostomy bag while large enough to serve as a back flow retention lip.

A third overall diameter D3 is shown to correspond to the outermost extent of the intermediate neck portion 328. The diameter D3 is less than both the diameters D1 and D2. D3 should be large enough to accommodate the stoma 340 but small enough to fit within the diameter of the central bag aperture (e.g., fourth overall diameter D4) so that the intermediate neck portion 328 can extend between and protect the sidewall 346 of the stoma 340 from the jagged edges of the bag opening.

In the illustrated example of FIG. 9, the neck portion diameter D3 is essentially equal to the bag aperture diameter D4, but D4 can be larger than D3, so long as D4 is smaller than D2. While not separately denoted in FIG. 9, it will be noted that the central plate aperture of the retention plate 330 (see 126 in FIG. 4A) also has a fifth diameter, which can be referred to as D5, which is equal to D3 and D4. As noted above with respect to the corner junction 147 in FIGS. 2A-2D, setting D3 equal to DS provides an additional sealing interface at the junction between the lower annular flange 326 and the intermediate neck portion 328.

FIG. 10 is another system 350 similar to the system 320 in FIG. 9. As such, like reference numerals have been used in FIG. 10 to identify similar elements as in FIG. 9. However, in this case a seal insert 322A includes a contoured, tapered intermediate neck portion 328A with a curvilinearly extending inner sidewall 344A that is sized and shaped to contactingly engage the outer sidewall 346 of the stoma 340. In this way, the seal insert 322A directly contacts and nominally seals against the stoma 340.

As before, this arrangement is intended to reduce leakage of the bag contents onto the peristomal area of the torso 338. Seal inserts with different shapes, sizes, radii of curvature, etc. can be provided for different sizes and shapes of stomas, so the respective diagrams of FIGS. 9-10 are merely exemplary and are not limiting.

It will now be appreciated that the various embodiments presented herein can provide a number of benefits over the existing art. The system eliminates the need to use adhesive, sealing or other material layers in direct contact with the peristomal area, reducing irritation and enhancing cleaning effectiveness. This provides a secure, non-adhesive seal between the drain pouch (bag) and the wearer.

The seal insert further provides protection of the stoma against the opening cut through the bag. This opening is scissor-cut by the user and can be rough and jagged, leading to irritation, infection and other effects. Bags with precut openings of any number of different sizes can be used as well.

The rolled back flow lip (back flow retention flange) provides protection against back flow of bag contents onto the wearer. The back flow lip also helps to retain the seal insert in the bag during installation of the appliance.

The system is comfortable for the user, and accommodates movement while maintaining the appliance securely affixed to the body. The system is low-profile and allows the bag to be concealed under clothing worn by the patient. The system can accommodate both vertical and horizontal orientations of the bag, and can work with pouches or other carrying mechanisms. While the various embodiments have used both the seal insert and a plate assembly with a belt to compress the seal insert against the torso of the patient, the seal insert can be utilized separately so long as compressive force is maintained against the torso of the patient using a suitable compression mechanism.

It is to be understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. An apparatus for supporting an ostomy bag of an ostomate having a stoma, the apparatus comprising:
a flexible seal insert comprising a first end, an opposing second end, and an intermediate neck portion extending therebetween to define a central passageway, the first end comprising a first annular flange extending radially about the central passageway and configured to contact a peristomal area of the ostimate for sealing compression thereagainst, the second end comprising a second annular flange configured for insertion through an opening of the ostomy bag to provide a back flow retention lip to facilitate retention of contents within the bag issued by the stoma, the first annular flange having a first overall diameter, the second annular flange having a second overall diameter less than the first overall diameter, and the intermediate neck portion having a third overall diameter less than the second overall diameter.

2. The apparatus of claim 1, further comprising:
a retention plate adapted to be secured to a torso of the ostimate using an encircling belt, the plate having a first planar surface, a second planar surface and a central plate aperture extending through a thickness of the plate from the first planar surface to the second planar surface, the central plate aperture sized to accommodate passage of the intermediate neck portion of the seal insert therethrough.

3. The apparatus of claim 2, wherein the first planar surface is configured for adhesive attachment to the ostomy bag using a layer of adhesive, and wherein the second planar surface is configured for pressing engagement against the first annular flange of the seal insert.

4. The apparatus of claim 1, wherein the intermediate neck portion is configured to surround the stoma and extend between the stoma and the central opening of the ostomy bag to physically isolate the stoma from an edge of the central opening of the ostomy bag.

5. The apparatus of claim 1, wherein the intermediate neck portion is sized to pass in non-contacting relation to an outer annular sidewall of the stoma.

6. The apparatus of claim 1 wherein the intermediate neck portion has an inner annular sidewall sized to pass in contacting, sealing relation to an outer annular sidewall of the stoma.

7. The apparatus of claim 1, further comprising a fluid barrier member configured to be interposed in contacting engagement between an inner annular sidewall of the intermediate neck portion and an outer annular sidewall of the stoma.

8. The apparatus of claim 1, further comprising an ostomy bag coupled to the seal insert and a belt which affixes the seal insert to a torso of the ostomate.

9. The apparatus of claim 8, wherein the ostomy bag is characterized as a one-piece ostomy pouch assembly, wherein the apparatus further comprises an intervening retention plate to which the one-piece ostomy pouch assembly is attached, the belt affixed to the plate, the plate compressingly engaging the first annular flange of the seal insert against the torso of the ostimate.

10. The apparatus of claim 8, wherein the ostomy bag is characterized as a two-piece ostomy pouch assembly comprising a pouch portion and a coupling portion, wherein the coupling portion is affixed directly to the first annular flange of the seal insert using a layer of adhesive, wherein the pouch portion is affixed to the coupling portion, and wherein the belt is affixed to the pouch portion.

11. A system, comprising:
an ostomy bag having a bag portion with a central bag aperture, an annular seal surrounding the central bag aperture, and an annular adhesive layer surrounding the annular seal;
a flexible seal insert comprising a first end, an opposing second end, and an intermediate neck portion extending therebetween to define a central passageway, the first end comprising a first annular flange extending radially about the central passageway and configured to contact a peristomal area of an ostomate for sealing compression thereagainst, the second end comprising a second annular flange configured for insertion through the central bag aperture to provide a back flow retention lip to facilitate retention of contents within the bag portion issued by the stoma; and
a retention plate adapted to be secured to a torso of the ostomate using an encircling belt, the plate having a first planar surface, a second planar surface and a central plate aperture extending through a thickness of the plate from the first planar surface to the second planar surface, the central plate aperture sized to accommodate passage of the intermediate neck portion of the seal insert therethrough, the annular seal and the annular adhesive layer contactingly engaging the first planar surface, the second planar surface contactingly compressing the first annular flange against the peristomal area;
wherein the first annular flange has a first overall diameter, the second annular flange has a second overall diameter less than the first overall diameter, the intermediate neck portion has a third overall diameter less than the second overall diameter, and the central bag aperture has a fourth overall diameter less than the second overall diameter and greater than or equal to the third overall diameter.

12. The system of claim 11, wherein the intermediate neck portion is configured to surround the stoma and extend between the stoma and the central bag aperture of the ostomy bag to physically isolate the stoma from an edge of the central bag aperture of the ostomy bag.

13. The system of claim 11, wherein the intermediate neck portion is sized to pass in non-contacting relation to an outer annular sidewall of the stoma.

14. The system of claim 11, wherein the intermediate neck portion has an inner annular sidewall sized to pass in contacting, sealing relation to an outer annular sidewall of the stoma.

15. The system of claim 11, further comprising a fluid barrier member interposed in contacting engagement between an inner annular sidewall of the intermediate neck portion and an outer annular sidewall of the stoma.

16. The system of claim 11, wherein the seal insert is formed of silicone.

17. The system of claim 11, wherein the first annular flange has a lower contact surface that extends along a frusto-conical path when disengaged from the peristomal area of the ostomate and which nominally lies flat against the peristomal area when the seal insert is compressed thereagainst by a plate member.

18. A method for attaching an ostomy bag to an ostomate, comprising:
providing an ostomy bag having a bag portion with a central bag aperture, an annular seal surrounding the central bag aperture, and an annular adhesive layer surrounding the annular seal;
adhering the bag portion to a retention plate using the annular adhesive layer, the retention plate having a central plate aperture aligned with the central bag aperture;
inserting a seal insert through the central plate aperture and the central bag aperture, the seal insert comprising a first end, an opposing second end, and an intermediate neck portion extending therebetween to define a central passageway, the first end comprising a first annular flange extending radially about the central passageway and configured to contact a peristomal area of the ostomate for sealing compression thereagainst, the second end comprising a second annular flange configured for insertion through the central bag aperture to provide a back flow retention lip to facilitate retention of contents within the bag portion issued by the stoma;
lowering the seal insert, the central plate and the ostomy bag onto the ostomate so that the stoma extends through the central passageway, the central plate aperture and the central bag aperture and so that the first annular flange contactingly engages the peristomal area; and
securing the retention plate to a torso of the ostomate, wherein the first annular flange has a first overall diameter, the second annular flange has a second overall diameter less than the first overall diameter, the intermediate neck portion has a third overall diameter less than the second overall diameter, and the central bag aperture has a fourth overall diameter less than the second overall diameter and greater than the third overall diameter.

19. The method of claim 18, wherein the securing step comprises using a belt attached to opposing ends of the central plate to secure the retention plate to the torso of the ostomate.

20. The method of claim 18, further comprising a prior step of cutting a web of material of the ostomy bag to form the central bag aperture using a cutting tool, wherein the intermediate neck portion extends between an edge of the central bag aperture and an outer annular sidewall of the stoma during the lowering and securing steps.
